(19) United States
(12) Patent Application Publication
(10) Pub. No.: US 2018/0132816 A1
(43) Pub. Date: May 17, 2018

(54) IMAGING SYSTEM AND METHOD FOR WIRE POSITIONING

(71) Applicants: Hui-Hua CHIANG, Taipei City (TW); METAL INDUSTRIES RESEARCH & DEVELOPMENT CENTRE, KAOHSIUNG (TW)

(72) Inventors: Ming-Hui CHEN, Kaohsiung City (TW); Wei-Te CHEN, Changhua County (TW); Hui-Hua CHIANG, Taipei City (TW)

(21) Appl. No.: 15/394,768

(22) Filed: Dec. 29, 2016

(30) Foreign Application Priority Data
Nov. 14, 2016 (TW) 105137123

(51) Int. Cl. A61B 8/08; A61B 8/12

(52) U.S. Cl. CPC A61B 8/0841; A61B 8/5246; A61B 8/5207; A61B 8/12

(57) ABSTRACT

The imaging system includes a wire, a second signal transmission unit and a computer system. At least one first signal transmission unit is disposed on the wire which is configured to enter a blood vessel in a human body. The second signal transmission unit is disposed outside of the human body. The computer system is electrically connected to the second signal transmission unit. The first signal transmission unit emits a first signal, the second signal transmission unit receives the first signal, and the computer system generates a first image according to the first signal. The second signal transmission unit emits a second signal, and receives a reflection signal corresponding to the second signal. The computer system generates a second image according to the reflection signal, and merges the first image with the second image as a third image for rendering a position of the wire in the human body.

### RELATED APPLICATIONS

This application claims priority to Taiwan Application Serial Number 105137123 filed Nov. 14, 2016, which is herein incorporated by reference.

### BACKGROUND

### Field of Invention

The present invention relates to an imaging method. More particularly, the present invention relates to a bi-directional imaging method for wire positioning.

### Description of Related Art

The World Health. Organization (WHO) predicts that more than twenty-three million people will die due to cardiovascular diseases annually in the world before year 2030. Ministry of health and welfare in Taiwan predicts that number of the patient suffered from cardiovascular diseases will increase 0.5 million per year. The number of patients having cardiovascular disease has been the highest in the world in an aspect of demand side of medical market. The product demand for all kinds of wire/catheter has been increased for years, and the scale of the global market is nearly 28.6 billion dollars in 2014, and is predicted to be 42.4 billion dollars in 2019. Factors driving this wire/catheter market includes increasing need for minimally invasive procedures, increasing elderly population, and diseases such as obesity caused by lifestyle which results in the grow of cardiovascular diseases.

Minimally invasive surgery with cardiac catheterization is a main treatment for cardiovascular diseases. Take invasive treatment of coronary heart disease as an example, an arterial sheath (the diameter thereof is about 2 mm to 3 mm) is plugged into an artery in arm or groin, and a steel wire enters a treatment part through the blood vessel to establish a surgery channel. An instrument combined with a plastic catheter enters the blood vessel through the wire for diagnosis and treatment. Therefore, putting the wire into the treatment location through the blood vessel is the first step, and problems in the surgery includes: unidirectional operation which needs many trying; lots of product specification based on the context; large amount of X ray and developer are required; depend on doctor's experience and high risk. The massive using of X ray and developer may cause other organs (e.g. kidney) damaged. Therefore, it is an issue in the art about how to devise an imaging method for tracking a wire in the human body to provide route guide during the surgery and decrease the amount the X ray and developer.

### SUMMARY

An objective of the invention is to provide an imaging method and an imaging system for wire positioning thereof, in which an angle and a route of the wire in the blood vessel could be guided based on bi-directional wire imaging.

Embodiments of the invention provide an imaging system for wire positioning. The imaging system includes a wire, a second signal transmission unit and a computer system. At least one first signal transmission unit is disposed on the wire, and the wire is configured to enter a blood vessel in a human body. The second signal transmission unit is disposed outside of the human body. The computer system is electrically connected to the second signal transmission unit. The first signal transmission unit emits a first signal, the second signal transmission unit receives the first signal, and the computer system generates a first image according to the first signal. The second signal transmission unit emits a second signal, and receives a reflection signal corresponding to the second signal. The computer system generates a second image according to the reflection signal, and merges the first image with the second image as a third image for rendering a position of the wire in the human body.

### BRIEF DESCRIPTION OF THE DRAWINGS

The invention can be more fully understood by reading the following detailed description of the embodiment, with reference made to the accompanying drawings as follows. The patent or application file contains at least one drawing executed in color. Copies of this patent or patent application publication with color drawing(s) will be provided by the Office upon request and payment of the necessary fee.

FIG. 1 is a schematic diagram illustrating an imaging system for wire positioning according to an embodiment.

FIG. 2 is a diagram illustrating calculation of position of the first signal transmission unit 111.

FIG. 3 is a diagram illustrating merging of the first image and the second image.

FIG. 4 is a schematic diagram illustrating the wire according to an embodiment.

FIG. 5 is a diagram illustrating an example of the third image according to an embodiment.

FIG. 6 is a diagram illustrating a flow chart of a wire positioning method according to an embodiment.

### DETAILED DESCRIPTION

Specific embodiments of the present invention are further described in detail below with reference to the accompanying drawings, however, the embodiments described are not intended to limit the present invention and it is not intended for the description of operation to limit the order of implementation. Moreover, any device with equivalent functions that is produced from a structure formed by a recombination of elements shall fall within the scope of the present invention. Additionally, the drawings are only illustrative and are not drawn to actual size.

The using of “first”, “second”, “third”, etc. in the specification should be understood for identifying units or data described by the same terminology, but are not referred to particular order or sequence. In addition, the term “couple” used in the specification should be understood for electrically connecting two units directly or indirectly. In other words, when “a first object is coupled to a second object” is written in the specification, it means another object may be disposed between the first object and the second object.

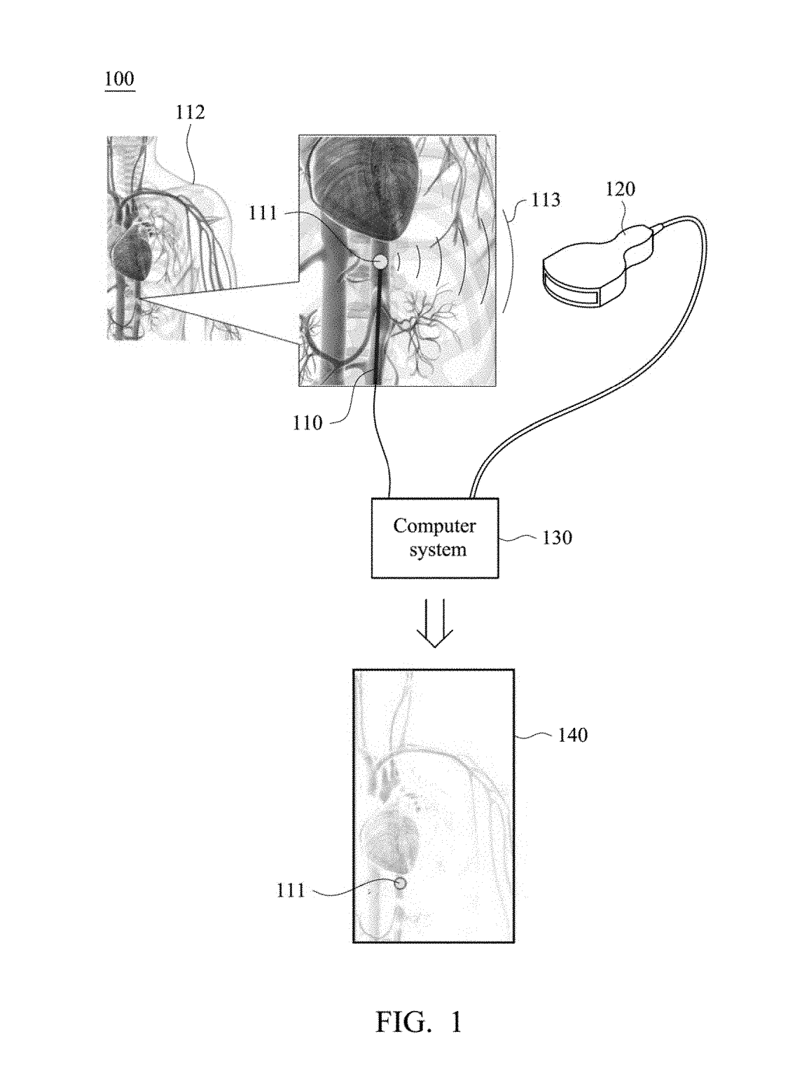

FIG. 1 is a schematic diagram illustrating an imaging system for wire positioning according to an embodiment. Referring to FIG. 1, an imaging system 100 includes a wire 10, a second signal transmission unit 120 and a computer system 130. The wire 110 is configured to enter a blood vessel of a human body 112. In some embodiments, the diameter of the wire 110 is greater than or equal to 0.5 millimeter and less than or equal to 2 millimeter, and the wire 110 may be made of metal or other biocompatible material. At least one first signal transmission unit 111 is disposed on the wire 110. The second signal transmission unit 120 is disposed outside of the human body 112. The computer system 130 is electrically connected to the second signal transmission unit 120 in a wire or wireless way.

In the imaging method provided in the disclosure, the first signal transmission unit 111 emits a first signal which is received by the second signal transmission unit 120. In addition, the second signal transmission unit 120 emits a second signal which is received by the second signal transmission unit 120 itself. The position of the wire in the human body may be clearly rendered through the bi-directional imaging. In the embodiment, the first signal transmission unit 111 is an ultrasound transducer, and the second signal transmission unit 120 is an ultrasound detector. In other words, ultrasound imaging is used to position the wire in the embodiment, and both of the first signal and the second signal are ultrasound signals. However, the invention is not limited thereto, other positioning approaches may be used in other embodiments, and the first signal transmission unit 111 and the second signal transmission unit 120 may be other corresponding devices.

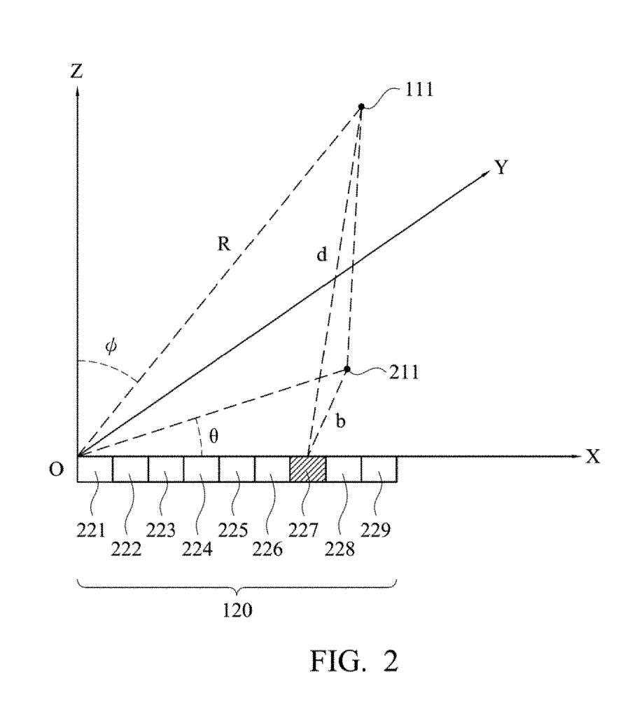

The operation of the imaging system 100 is described below. The first signal transmission unit 111 emits the first signal 113 (e.g. ultrasound signal), and the second signal transmission unit 120 receives the first signal 113. Multiple ultrasound transducers are disposed in the second signal transmission unit 120, and the position of the first signal transmission unit 111 relative to the second signal transmission unit 120 can be calculated according to time difference of the ultrasound transducers receiving the first signal 113. To be specific, referring to FIG. 2, polar coordinates are used to describe the position of the first signal transmission unit 111. R is a distance between the first signal transmission unit 111 and an original point O. φ is an angle of the first signal transmission unit 111 relative to a Z axis. A coordinate point 211 is a projection of the first signal transmission unit 111 on an X-Y plane. Θ is an angle of the coordinate point 211 relative to an X axis. N ultrasound transducers (e.g. ultrasound transducers 221-229) are disposed on the X axis, and the ultrasound transducers 221-229 are disposed in the second signal transmission unit 120. Take the ultrasound transducer 227 as an example, the distance between the first signal transmission unit 111 and the ultrasound transducer 227 is d which is represented by a following equation (1).

$$\begin{matrix}{d}^{2} = {( R cos ϕ )}^{2} + {b}^{2} \\ = {R}^{2} {cos}^{2} ϕ + {( R sin ϕ sin θ )}^{2} + {( R sin ϕ cos θ - {x}_{i} )}^{2} \\ = {R}^{2} {cos}^{2} ϕ + {R}^{2} {sin}^{2} ϕ {sin}^{2} θ + {R}^{2} {sin}^{2} ϕ {cos}^{2} θ + {x}_{i}^{2} - 2 R sin ϕ cos θ \\ = {R}^{2} + {x}_{i}^{2} - 2 R sin ϕ cos θ\end{matrix} \qquad ( 1 )$$

b is the distance between the coordinate point 211 and the ultrasound transducer 227. xᵢ is the distance between the ultrasound transducer 227 and the original point O. Therefore, the time for the ultrasound signal propagating from the first signal transmission unit 111 to the ultrasound transducer 227 is tᵢ which is represented by a following equation (2).

$${t}_{i} = \frac{\sqrt{{R}^{2} + {x}_{i}^{2} - 2 R sin ϕ cos θ}}{c} \qquad ( 2 )$$

c is the velocity of the ultrasound signal. In general, the velocity of the ultrasound signal in different tissues of human body are basically the same, and thus the velocity c could be constant. However, there may be air between the first signal transmission unit 111 and the ultrasound transducer 227, and the velocity of the ultrasound signal in the air is different from that in the tissues of human body. Therefore, after considering the effect of air, the equation (2) is modified as a following equation (3).

$${t}_{i} = \frac{\sqrt{{R}^{2} + {x}_{i}^{2} - 2 R sin ϕ cos θ} - y}{{c}_{1}} + \frac{y}{{c}_{2}} \qquad ( 3 )$$

y is the distance that the ultrasound signal propagates in air. The distance y is greater or equal to 0 and less than the distance d. c₁ is the velocity of the ultrasound signal propagating in a tissue of human body, and c₂ is the velocity of the ultrasound signal propagating in the air. The two velocities c₁, c₂ should be known. Note that the distance R, the angle φ, the angle θ, and the distance y are unknown.

On the other hand, after the ultrasound transducers 221-229 receive the ultrasound signal, the practical time of the ultrasound signal transmitted to the ultrasound transducers 221-229 can be obtained and herein is respectively denoted as τ₁ . . . τ_{N}, where N is a positive integer. Next, a following optimization algorithm is performed to obtain the unknown variables.

$$\begin{matrix}{min}_{R , ϕ , θ , y} {\sum }_{i}^{ } {( \frac{\sqrt{{R}^{2} + {x}_{i}^{2} - 2 R sin ϕ cos θ} - y}{{c}_{1}} + \frac{y}{{c}_{2}} - {τ}_{i} )}^{2} & ( 4 ) \\ s . b . 0 \leq y \leq \sqrt{{R}^{2} + {x}_{i}^{2} - 2 R sin ϕ cos θ} & ( 5 )\end{matrix}$$

The equation (4) is an objective function, and the equation (5) is the constraint. The positive integer i is from 1 to N (corresponding to the ultrasound transducers 221-229). In other words, the equation (4) is to calculate the optimal distance R, the angle φ, the angle θ, and the distance y so that the difference between the estimated time tᵢ and the practical time τᵢ is minimized. Because the equation (4) has four variables, the number (i.e. the positive number N) of the ultrasound transducers 221-229 has to be greater or equal to 4.

Referring to FIG. 1 again, after the distance R, the angle φ and the angle θ are obtained, the position of the first signal transmission unit 111 relative to the second signal transmission unit 120 is obtained. The computer system 130 would generate a first image 140, in which the position of the first signal transmission unit 111 is rendered. Note that the first image 140 is just a schematic diagram, and tissues in the human body are not rendered in the first image 140.

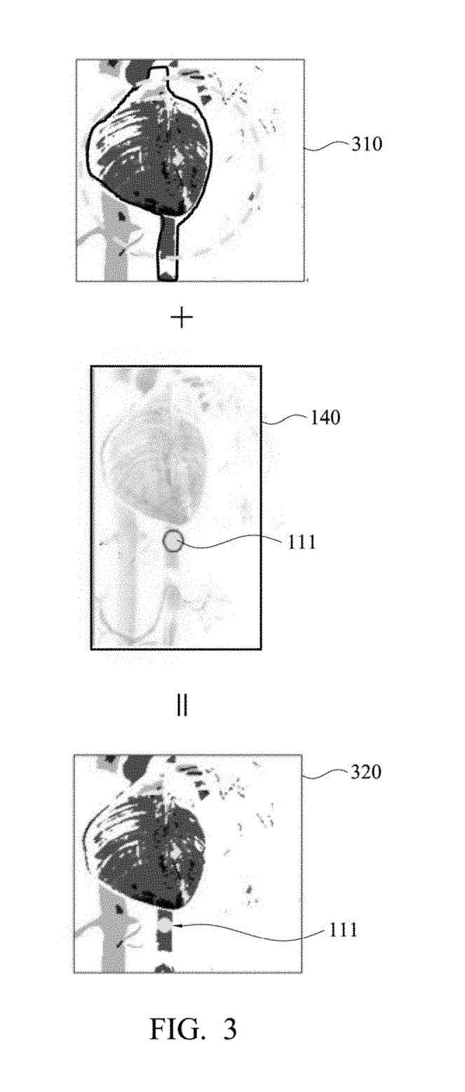

Next, referring to FIG. 1 and FIG. 3, the second signal transmission unit 120 emits a second signal (e.g. ultrasound signal) toward the tissues in the human body, and the reflected ultrasound signal is called a reflection signal. The second signal transmission unit 120 receives the reflection signal, and the computer system 130 generate a second image 310. In other words, the second image 310 is obtained by conventional ultrasound imaging which may be three-dimensional imaging. In the conventional imaging, the position of the blood vessel cannot be clearly recognized via the second image 310. However, in the embodiment, the computer system 130 would merge the first image 140 with the second image 310 as a third image 320 to render the position of the wire 110 in the human body. For example, the position of the first signal transmission unit 111 is obtained from the first image 140, and the position of the first signal transmission unit 111 is rendered in the second image 310. Consequently, the position of the wire 110 is rendered because the first signal transmission unit 111 is disposed on the wire 110.

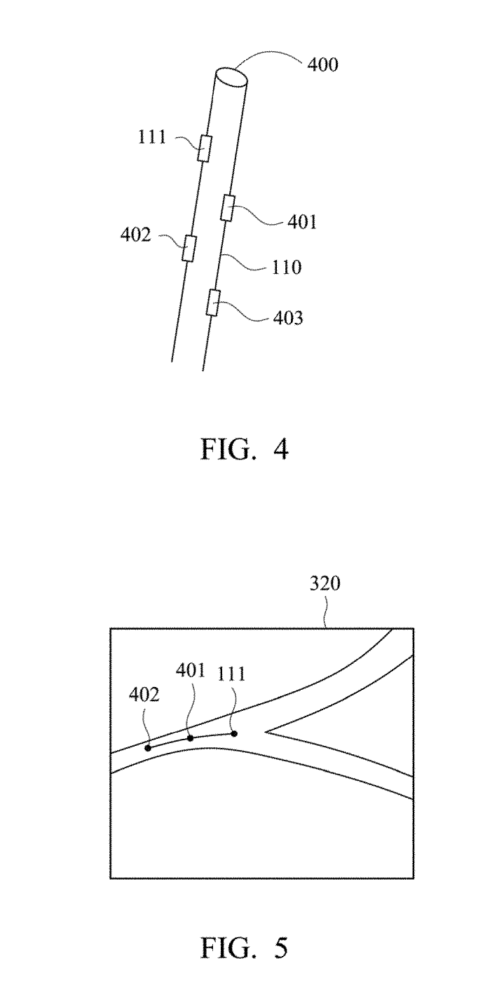

FIG. 4 is a schematic diagram illustrating the wire according to an embodiment. Referring to FIG. 4, first signal transmission units 111 and 401-403 are disposed on the wire 110 in some embodiments. The first signal transmission units 111 and 401-403 are disposed along with the wire 110. That is, distances between a first terminal 400 of the wire 110 and the first signal transmission units 111, 401-403 are different from each other. Each of the first signal transmission units 111 and 401-403 would emit the aforementioned ultrasound signal to the second signal transmission unit 120 to obtain the positions of the first signal transmission units 111 and 401-403 relative to the second signal transmission unit 120. As a result, referring to FIG. 5, the positions of the first signal transmission units 111, 401 and 402 may be rendered in the third image 320. In some embodiments, only the positions of the first signal transmission units 111, 401 and 402 are rendered. That is, each of the first signal transmission units 111, 401 and 402 may be rendered as a point. However, in other embodiments, connections between the first signal transmission units 111, 401 and 402 may be rendered to represent the wire 110. For example, in the embodiment of FIG. 5, the wire 110 is facing a branch of the blood vessel, but the healthcare provide is capable of knowing where the wire 110 is through the connection between the first signal transmission units 111, 401 and 402.

In the embodiment, both of the first signal and the second signal are ultrasound signals, and frequencies of both of them are less than 10 megahertz because low-frequency ultrasound signals have high transmittance. However, the frequencies of the ultrasound signals may be higher than 10 megahertz in some embodiments, and the invention is not limited thereto.

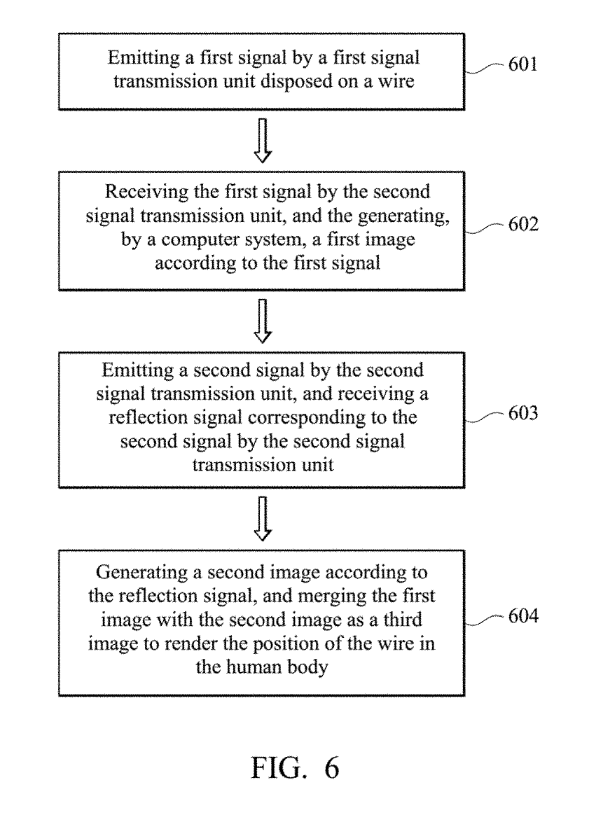

FIG. 6 is a diagram illustrating a flow chart of an imaging method for wire positioning according to an embodiment. In step 601, a first signal is emitted by a first signal transmission unit disposed on a wire. In step 602, the first signal is received by the second signal transmission unit, and the computer system generates a first image according to the first signal. In step 603, a second signal is emitted by the second signal transmission unit, and a reflection signal corresponding to the second signal is received by the second signal transmission unit. In 604, a second image is generated according to the reflection signal, and the first image is merged with the second image as a third image to render the position of the wire in the human body. However, each step in FIG. 6 has been described in detail above, and therefore the description will not be repeated. Note that the steps in FIG. 6 may be implemented as programs or circuits, which is not limited in the invention. In addition, the method of FIG. 6 may be performed with the aforementioned embodiments or independently. In other words, other steps may be inserted between the steps of FIG. 6.

Although the present invention has been described in considerable detail with reference to certain embodiments thereof, other embodiments are possible. Therefore, the spirit and scope of the appended claims should not be limited to the description of the embodiments contained herein. It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. An imaging system for wire positioning, wherein the imaging system comprises:
a wire, wherein at least one first signal transmission unit is disposed on the wire, and the wire is configured to enter a blood vessel in a human body;
a second signal transmission unit, disposed outside of the human body; and
a computer system electrically connected to the second signal transmission unit,
wherein the at least one first signal transmission unit emits a first signal, the second signal transmission unit receives the first signal, and the computer system generates a first image according to the first signal,
wherein the second signal transmission unit emits a second signal, and receives a reflection signal corresponding to the second signal,
wherein the computer system generates a second image according to the reflection signal, and merges the first image with the second image as a third image for rendering a position of the wire in the human body.

2. The imaging system of claim 1, wherein a number of the at least one first signal transmission unit is greater than one, and distances between a first terminal of the wire and the first signal transmission units are different from each other.

3. The imaging system of claim 2, wherein the computer system renders a connection between at least two of the first signal transmission units in the third image.

4. The imaging system of claim 1, wherein the first signal and the second signal are ultrasound signals, and frequencies of the first signal and the second signal are less than 10 megahertz.

5. The imaging system of claim 1, wherein a diameter of the wire is greater than 0.5 millimeter and equal or less than 2 millimeter.

6. The imaging system of claim 1, wherein the first signal is an ultrasound signal, the at least one first signal transmission unit is an ultrasound transducer, the second signal transmission unit is an ultrasound detector, and the operation of the computer system generating the first image according to the first signal comprises:
the computer system performs following equations (1) and (2) to calculate a position of the ultrasound transducer relative to the ultrasound detector,
$$\begin{matrix}{min}_{R , ϕ , θ , y} {\sum }_{i}^{ } {( \frac{\sqrt{{R}^{2} + {x}_{i}^{2} - 2 R sin ϕ cos θ} - y}{{c}_{1}} + \frac{y}{{c}_{2}} - {τ}_{i} )}^{2} & ( 1 ) \\ s . b . 0 \leq y \leq \sqrt{{R}^{2} + {x}_{i}^{2} - 2 R sin ϕ cos θ} , & ( 2 )\end{matrix}$$
wherein R is a distance between the ultrasound transducer and an original point, φ is an angle of the ultrasound transducer relative to a Z axis, a coordinate point is projection of the ultrasound transducer on a X-Y plane, θ is an angle of the coordinate point relative to a X axis, y is a distance that the ultrasound signal propagates in air, xᵢ is a distance between an i^{th} ultrasound transducer of a plurality of ultrasound transducers in the ultrasound detector and the original point, c₁ is a velocity of the ultrasound signal propagating in an tissue, c₂ is a velocity of the ultrasound signal propagating in the air, and τᵢ is time of the ultrasound signal propagating from the ultrasound transducer to the i^{th} ultrasound transducer.

7. An imaging method for a computer system, wherein the imaging method comprises:
emitting, by at least one first signal transmission unit, a first signal, wherein the at least one first signal transmission unit is disposed on a wire which is configured to enter a blood vessel in a human body;
receiving, by a second signal transmission unit, the first signal, and generating, by the computer system, a first image according to the first signal, wherein the second signal transmission unit is disposed outside of the human body;
emitting, by the second signal transmission unit, a second signal, and receiving, by the second signal transmission unit, a reflection signal corresponding to the second signal; and
generating a second image according to the reflection signal, and merging the first image with the second image as a third image for rendering a position of the wire in the human body.

8. The imaging method of claim 7, wherein a number of the at least one first signal transmission unit is greater than one, and the imaging method further comprises:
rendering a connection between at least two of the first signal transmission units in the third image.

9. The imaging method of claim 7, wherein the first signal and the second sign are ultrasound signals, and frequencies of the first signal and the second signal are less than 10 megahertz.

10. The imaging method of claim 7, wherein the first signal is an ultrasound signal, the at least one first signal transmission unit is an ultrasound transducer, the second signal transmission unit is an ultrasound detector, and the imaging method further comprises:
performing following equations (1) and (2) to calculate a position of the ultrasound transducer relative to the ultrasound transducer,
$$\begin{matrix}{min}_{R , ϕ , θ , y} {\sum }_{i}^{ } {( \frac{\sqrt{{R}^{2} + {x}_{i}^{2} - 2 R sin ϕ cos θ} - y}{{c}_{1}} + \frac{y}{{c}_{2}} - {τ}_{i} )}^{2} & ( 1 ) \\ s . b . 0 \leq y \leq \sqrt{{R}^{2} + {x}_{i}^{2} - 2 R sin ϕ cos θ} & ( 2 )\end{matrix}$$
wherein R is a distance between the ultrasound transducer and an original point, φ is an angle of the ultrasound transducer relative to a Z axis, a coordinate point is projection of the ultrasound transducer on a X-Y plane, θ is an angle of the coordinate point relative to a X axis, y is a distance that the ultrasound signal propagates in air, xᵢ is a distance between an i^{th} ultrasound transducer of a plurality of ultrasound transducers in the ultrasound detector and the original point, c₁ is a velocity of the ultrasound signal propagating in an tissue, c₂ is a velocity of the ultrasound signal propagating in the air, and τᵢ is time of the ultrasound signal propagating from the ultrasound transducer to the i^{th} ultrasound transducer.
